(19) United States
(12) Patent Application Publication
(10) Pub. No.: US 2018/0318001 A1
(43) Pub. Date: Nov. 8, 2018

(54) ELECTROSURGICAL INSTRUMENT

(71) Applicant: Gyrus Medical Limited, Cardiff (GB)

(72) Inventor: Marno Nagtegaal, Cardiff (GB)

(21) Appl. No.: 15/587,358

(22) Filed: May 4, 2017

(51) Int. Cl. A61B 18/14; A61B 18/12

(52) U.S. Cl. CPC A61B 18/1482; A61B 2018/0063; A61B 18/12; A61B 18/1447

(57) ABSTRACT

An electrode (1) for a jawed electrosurgical instrument has an elongate tissue-contacting surface (2), and a connection portion (8) comprising a crimp portion (12) and a support portion (9). The crimp portion (12) comprises a plate (10) which can be folded over the end of a lead (11) to form an electrical connection between the lead and the electrode. The support portion (9) forms an electrical connection between the crimp portion and the electrode, and is shaped and/or oriented such that the crimp portion (12) lies directly underneath the tissue contacting surface (2). The position of the connection portion (8) allows for it, including the crimp portion (12), to be encapsulated within an overmoulded polymer component (16).

### BACKGROUND OF THE INVENTION

This invention relates to a jawed electrosurgical instrument, to an electrode for such an instrument, and to a method for manufacturing a jaw member for an electrosurgical instrument, particularly a forceps instrument for sealing tissue. Such systems are commonly used for the treatment of tissue in surgical intervention, most commonly in “keyhole” or minimally invasive surgery, but also in “open” surgery.

It is known to manufacture jaw members for electrosurgical instruments by an overmoulding process, in which components are assembled into a mould and flowable material is introduced into the mould to surround the components and form the completed jaw member. U.S. Pat. No. 7,150,097 & 7,922,953 are examples of such manufacturing methods. It is also known to attach a lead to a conductive plate by means of crimping, and Australian patent application 2015264858 is one example of this type of connection.

### SUMMARY OF THE INVENTION

The present invention attempts to provide an improved alternative to crimped connections such as that illustrated in AU 2015264858. Accordingly, an electrode is provided for a jawed electrosurgical instrument, the electrode having a longitudinal axis extending from a proximal end of the electrode to a distal end of the electrode, the electrode having an elongate tissue-contacting surface, and a connection portion comprising a crimp portion and a support portion, the crimp portion comprising a plate which can be folded over the end of a lead to form an electrical connection between the lead and the electrode, the support portion forming an electrical connection between the crimp portion and the electrode, the support portion being shaped and/or oriented such that the crimp portion lies directly underneath the tissue-contacting surface at the proximal end of the electrode, such that the lead can emerge from the crimp portion in a proximal direction.

By orienting the crimp portion such that it lies directly underneath the tissue-contacting surface, there is less opportunity for the electrical voltage to short across to other electrically conductive components. In a convenient arrangement, the support portion is shaped and/or oriented such that the crimp portion lies at a specified angle to the tissue-contacting surface, typically parallel thereto. The support portion is conceivably shaped and/or oriented such that the distal end of the crimp portion faces the distal end of the electrode.

The connection portion is conveniently located at the proximal end of the tissue-contacting surface. In one convenient arrangement, the support portion has two 90° bends, such that it forms a U-shape (on its side) so as to place the crimp portion underneath the tissue-contacting surface. Preferably, the connection portion, including the crimp portion, is encapsulated within an overmoulded polymer component.

Placing the crimp portion underneath the tissue-contacting surface makes it easier to encapsulate the crimp portion within the overmoulded polymer, which typically forms part of a sub-component including the electrode, but leaving the tissue-contacting surface exposed from the overmoulded polymer. Encapsulating the crimp portion within the overmoulded polymer further prevents any electrical shorting between the crimp portion and any other electrically conductive components.

The invention further resides in an electrosurgical instrument including:

a handle including an actuating mechanism movable between a first position and a second position,

a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween,

a first sealing electrode located on the first jaw member, the first sealing electrode having a longitudinal axis extending from a proximal end of the electrode to a distal end of the electrode,

a second sealing electrode located on the second jaw member,

electrical connections capable of connecting the instrument to an electrosurgical generator, such that when the jaw members are in their closed position with tissue grasped therebetween, the instrument is capable of sealing the tissue by passing an electrosurgical current into the tissue from the first and second sealing electrodes,

the electrical connection for at least the first sealing electrode including a connection portion comprising a crimp portion and a support portion, the crimp portion comprising a plate which can be folded over the end of a lead to form an electrical connection between the lead and the first sealing electrode, the support portion forming an electrical connection between the crimp portion and the first sealing electrode, the support portion being shaped and/or oriented such that the crimp portion lies directly underneath the first sealing electrode with the lead emerging from the crimp portion in a proximal direction.

As before the support portion is shaped and/or oriented such that the crimp portion lies directly underneath the first sealing electrode, preferably such that the crimp portion lies parallel to the first sealing electrode, and conceivably such that the distal end of the crimp portion faces the distal end of the first sealing electrode. The connection portion is conveniently located at the proximal end of the first sealing electrode, and the connection portion, including the crimp portion, is preferably encapsulated within an overmoulded polymer component.

The invention also resides in a method for manufacturing a jaw member for an electrosurgical instrument, including the steps of:

providing an electrically conductive plate, the plate having a longitudinal axis extending from a proximal end of the plate to a distal end of the plate, the plate including an elongate tissue-contacting surface, and a connection portion comprising a crimp portion and a support portion;

introducing an electric lead in a distal direction such that an end portion of the lead overlaps the crimp portion with the lead emerging from the crimp portion in a proximal direction;

folding the crimp portion around the end portion of the lead to crimp the lead to the connection portion;

placing the electrically conductive plate into a first mould;

injecting a first flowable insulating material into the mould, such that the flowable material at least partially encapsulates the connection portion, including the crimp portion; and

allowing the first flowable insulating material to solidify to form a jaw component with the tissue-contacting surface exposed for contacting tissue.

The support portion is conveniently shaped and/or oriented such that the crimp portion lies directly underneath the tissue contacting surface. The method conveniently includes the further steps of

providing a jaw housing having a longitudinal jaw section;

placing the jaw housing and the jaw component into a second mould;

injecting a second flowable insulating material into the mould to secure the jaw component to the jaw housing;

allowing the second flowable insulating material to solidify; and

removing the jaw housing, jaw component and the solidified insulating material from the mould as a jaw member.

In this way, the crimp portion is encapsulated during a first overmoulding step, and the resulting subcomponent is further encapsulated during a second overmoulding step to form the final jaw assembly.

### BRIEF DESCRIPTION OF THE DRAWINGS

The invention will now be further described, by way of example only, with reference to the accompanying drawings, in which:

FIG. 1 is a schematic perspective view of an electrically conductive plate used in connection with the present invention,

FIG. 2 is a transparent perspective view of the electrically conductive plate of FIG. 1, subject to a first overmoulding process to form a jaw sub-component, and

FIG. 3 is a sectional perspective view of the jaw sub-component of

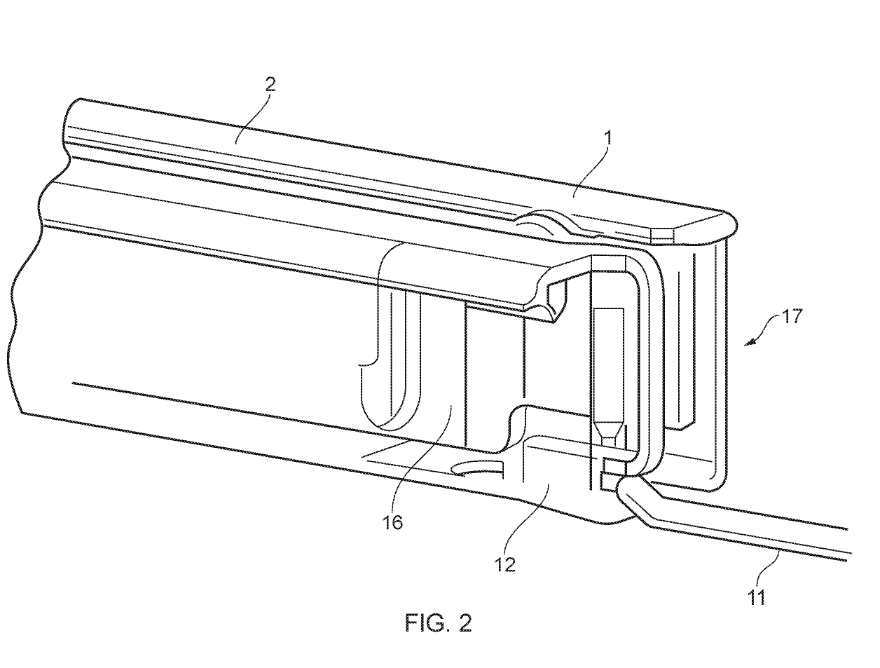

FIG. 2, subject to a further overmoulding process to for a jaw assembly.

### DETAILED DESCRIPTION OF EXEMPLARY EMBODIMENTS

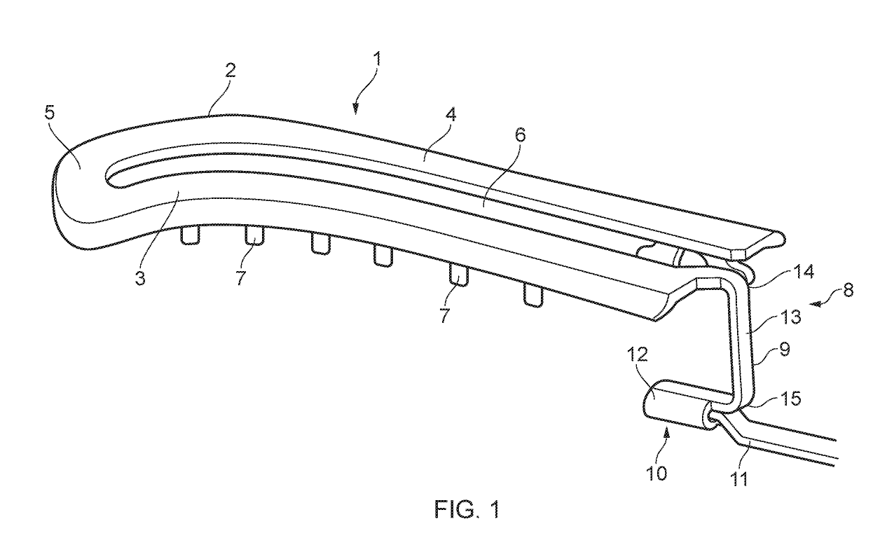

Referring to FIG. 1, an electrically conductive plate in the form of a shim 1 is manufactured from one or more of the processes of stamping, machining etching, folding or by a casting process, and comprises an upper surface 2 including a left sealing surface 3, a right sealing surface 4, and an end portion 5. The shim has a central slot 6, and a plurality of retaining tabs 7 are present at the edges of the shim 1, the retaining tabs being equally spaced along the sides of the shim. At the proximal end of the shim 1, opposite from the end portion 5, is a connection portion 8 consisting of a support 9 and a crimp plate 10. The crimp plate 10 is shown in FIG. 1 folded around the end of an electrical lead 11 to form a crimped electrical connection 12.

The support 9 is integrally formed with the proximal end of left sealing surface 3, and includes a straight section 13 bordered at each end by a first 90° bend 14 and a second 90° bend 15. The crimp plate 10 is located at the distal end of the support 9 such that the crimped connection 12 is located directly underneath the upper surface 2. This location protects the crimped connection from damage, and reduces the chances of shorting occurring between the crimped connection 12 and other electrically conductive components of the electrosurgical instrument.

FIG. 2 shows the shim 1, with the lead 11 having been connected by means of the crimped connection 12, encapsulated in an overmoulded polymer 16 to form a sub-component shown generally at 17. The overmoulded polymer 16 leaves the upper surface 2 exposed, but completely encapsulates the crimped connection 12 so as to eliminate the opportunity for electrical shorting to other conductive components.

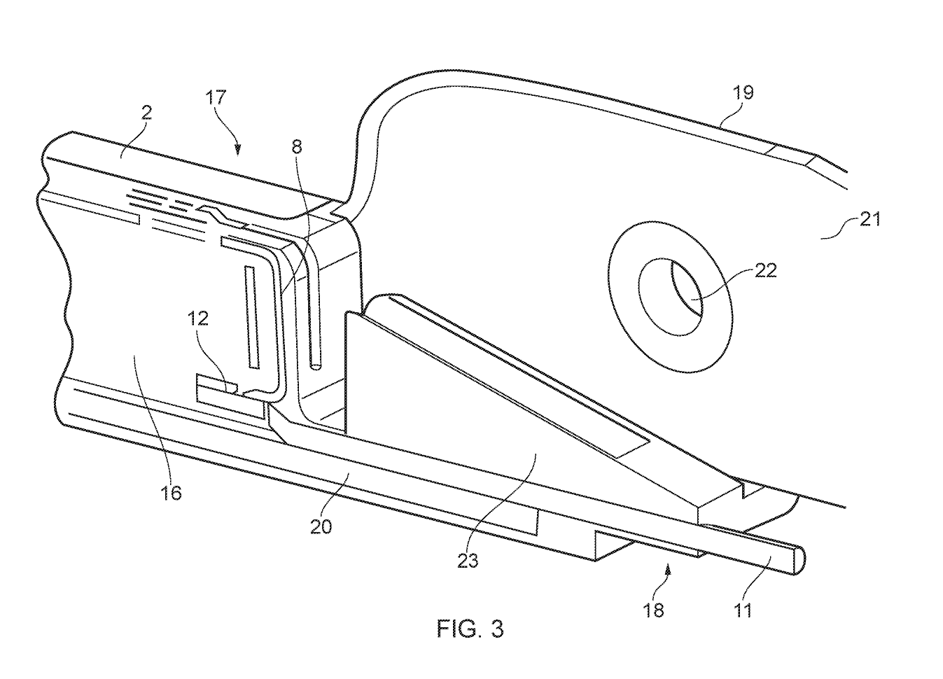

FIG. 3 shows the sub-component 17 having been subjected to a second overmoulding step in order to form a jaw assembly shown generally at 18. The subcomponent 17 is placed on a metallic jaw housing 19 which includes a base 20, a flange 21 and a pivot hole 22. The second overmoulding step introduces a second polymer 23 which secures the sub-component 17 to the jaw housing 19 and creates the jaw assembly 18. The lead 11 is further secured within the jaw assembly, protecting the crimped connection 12 from wear and damage.

Those skilled in the art will appreciate that modifications to the above can be made without departing from the scope of the present invention, and that alternative configurations of components can be employed. By positioning the crimped connection 12 underneath the upper surface 2, it is protected from damage and positioned so as to be encapsulated within the overmoulded polymer 16. A second overmoulding further protects the crimped connection, reducing any opportunity for shorting to occur.

What is claimed is:

1. An electrode for a jawed electrosurgical instrument, the electrode having a longitudinal axis extending from a proximal end of the electrode to a distal end of the electrode, the electrode having an elongate tissue-contacting surface, and a connection portion comprising a crimp portion and a support portion, the crimp portion comprising a plate which can be folded over the end of a lead to form an electrical connection between the lead and the electrode, the support portion forming an electrical connection between the crimp portion and the electrode, the support portion being shaped and/or oriented such that the crimp portion lies directly underneath the tissue contacting surface at the proximal end of the electrode, such that the lead can emerge from the crimp portion in a proximal direction.

2. An electrode according to claim 1, wherein the support portion is shaped and/or oriented such that the crimp portion lies at a specified angle to the tissue-contacting surface.

3. An electrode according to claim 2, wherein the support portion is shaped and/or oriented such that the crimp portion lies parallel to the tissue-contacting surface.

4. An electrode according to claim 2, wherein the support portion is shaped and/or oriented such that the distal end of the crimp portion faces the distal end of the electrode.

5. An electrode according to claim 1, wherein the connection portion is located at the proximal end of the tissue-contacting surface.

6. An electrode according to claim 1, wherein the connection portion, including the crimp portion, is encapsulated within an overmoulded polymer component.

7. An electrosurgical instrument including
a handle including an actuating mechanism movable between a first position and a second position,
a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween,
a first sealing electrode located on the first jaw member, the first sealing electrode having a longitudinal axis extending from a proximal end of the electrode to a distal end of the electrode,
a second sealing electrode located on the second jaw member,
electrical connections capable of connecting the instrument to an electrosurgical generator, such that when the jaw members are in their closed position with tissue grasped therebetween, the instrument is capable of sealing the tissue by passing an electrosurgical current into the tissue from the first and second sealing electrodes,
the electrical connection for at least the first sealing electrode including a connection portion comprising a crimp portion and a support portion, the crimp portion comprising a plate which can be folded over the end of a lead to form an electrical connection between the lead and the first sealing electrode, the support portion forming an electrical connection between the crimp portion and the first sealing electrode, the support portion being shaped and/or oriented such that the crimp portion lies directly underneath the first sealing electrode with the lead emerging from the crimp portion in a proximal direction.

8. An electrosurgical instrument according to claim 7, wherein the support portion is shaped and/or oriented such that the crimp portion lies at a specified angle to the first sealing electrode.

9. An electrosurgical instrument according to claim 7, wherein the support portion is shaped and/or oriented such that the crimp portion lies parallel to the first sealing electrode.

10. An electrosurgical instrument according to claim 7, wherein the support portion is shaped and/or oriented such that the distal end of the crimp portion faces the distal end of the first sealing electrode.

11. An electrosurgical instrument according to claim 7, wherein the connection portion is located at the proximal end of the first sealing electrode.

12. An electrosurgical instrument according to claim 7, wherein the connection portion, including the crimp portion, is encapsulated within an overmoulded polymer component.

13. An electrosurgical instrument according to claim 7, wherein the electrical connection for both the first and second sealing electrodes includes a connection portion comprising a crimp portion and a support portion, the support portions for each connections being shaped and/or oriented such that the crimp portion lies underneath its respective sealing electrode.

14. A method for manufacturing a jaw member for an electrosurgical instrument, including the steps of:
providing an electrically conductive plate, the plate having a longitudinal axis extending from a proximal end of the plate to a distal end of the plate, the plate including an elongate tissue-contacting surface, and a connection portion comprising a crimp portion and a support portion,
introducing an electric lead in a distal direction such that an end portion of the lead overlaps the crimp portion with the lead emerging from the crimp portion in a proximal direction;
folding the crimp portion around the end portion of the lead to crimp the lead to the connection portion;
placing the electrically conductive plate into a first mould;
injecting a first flowable insulating material into the mould, such that the flowable material at least partially encapsulates the connection portion, including the crimp portion, and
allowing the first flowable insulating material to solidify to form a jaw component with the tissue-contacting surface exposed for contacting tissue.

15. A method according to claim 14, wherein the support portion is shaped and/or oriented such that the crimp portion lies directly underneath the tissue contacting surface.

16. A method according to claim 14, including the further steps of
providing a jaw housing having a longitudinal jaw section;
placing the jaw housing and the jaw component into a second mould;
injecting a second flowable insulating material into the mould to secure the jaw component to the jaw housing;
allowing the second flowable insulating material to solidify; and
removing the jaw housing, jaw component and the solidified insulating material from the mould as a jaw member.
